(19) United States
(12) Patent Application Publication
(10) Pub. No.: US 2026/0253354 A1
(43) Pub. Date: Aug. 27, 2026

(54) SYSTEM AND METHOD FOR INTERACTIVE THREE-DIMENSIONAL VISUALIZATION OF PHYSIOLOGICAL STRESS EFFECTS

(71) Applicant: Morgan State Univesity, Baltimore, MD (US)

(72) Inventor: Stacey P. Patton, Baltimore, MD (US)

(21) Appl. No.: 19/535,329

(22) Filed: Feb. 10, 2026

Related U.S. Application Data
(60) Provisional application No. 63/756,315, filed on Feb. 10, 2025.

(51) Int. Cl. G06T 19/20; A61B 5/00; A61B 5/16; G06T 7/12; G06T 13/40; G06T 15/08; G06T 17/20; G16H 30/20; H04S 7/00

(52) U.S. Cl. CPC G06T 19/20; A61B 5/165; A61B 5/7405; A61B 5/742; G06T 7/12; G06T 13/40; G06T 15/08; G06T 17/20; G16H 30/20; A61B 2503/06; G06T 2200/24; G06T 2207/30004; G06T 2207/30204; G06T 2210/32; G06T 2210/36; G06T 2210/41; G06T 2210/62; G06T 2219/2012; G06T 2219/2016; G06T 2219/2021; H04S 7/304

(57) ABSTRACT

A system and method for interactive three-dimensional visualization of physiological stress effects on the human body uses biomarker correlation algorithms to determine physiological responses to physical trauma and toxic stress events. The system receives stress event data, calculates biomarker levels including cortisol, blood pressure, blood sugar, and inflammatory markers, and identifies affected organ systems including cardiovascular, nervous, endocrine, digestive, immune, and reproductive systems. Three-dimensional anatomical models are modified with visual characteristics depicting stress-induced changes based on the biomarker data, including color alterations, geometric deformations, and animated effects. A temporal progression interface enables visualization of immediate, short-term, and long-term physiological effects. Spatial audio synchronized with visual animations provides immersive auditory feedback. Cross-platform rendering modules optimize visualization for mobile devices, desktop computers, tablets, virtual reality headsets, and augmented reality glasses. The system provides for real-time interactive manipulation of anatomical visualizations, particularly for pediatric toxic stress education related to corporal punishment.

### CROSS-REFERENCE TO RELATED APPLICATIONS

This application claims priority to U.S. Provisional Patent Application No. 63/756,315, filed Feb. 10, 2025, entitled “Toxic Stress Modeling Method,” the specification of which is incorporated herein by reference in its entirety.

### FIELD OF THE INVENTION

The present invention relates to medical visualization systems and methods, and more particularly to interactive three-dimensional visualization systems for depicting physiological effects of physical trauma and toxic stress on the human body using extended reality technologies.

### BACKGROUND

Medical education and patient communication regarding the internal physiological effects of physical trauma, particularly corporal punishment in pediatric populations, face significant challenges. Traditional educational materials such as static images, pamphlets, and two-dimensional diagrams fail to convey the complex, systemic nature of toxic stress responses within the human body. Healthcare professionals, including pediatricians, child protective services workers, educators, and social workers, lack effective tools to communicate the invisible internal damage caused by physical punishment to parents, caregivers, and other stakeholders.

Toxic stress, particularly in children, results from repeated activation of stress response systems without adequate support or protection. Physical punishment triggers cascading physiological responses involving multiple organ systems, including the cardiovascular, nervous, endocrine, digestive, immune, and reproductive systems. These responses involve complex biomarker changes, including alterations in cortisol levels, blood pressure, blood sugar levels, and inflammatory markers, which can lead to both immediate and long-term health consequences.

Several prior art systems have addressed medical visualization and training. U.S. Pat. No. 12,308,099 describes an extended reality medical report generating system that creates patient-specific virtual tours of gastrointestinal findings by pinning discrete conditions to anatomical diagrams and painting diffuse conditions onto body segments. U.S. Pat. No. 11,694,328 discloses methods for outputting augmented reality information during medical examinations, including overlaying anatomical structure markings on patients using machine learning algorithms. U.S. Pat. No. 11,557,216 teaches an adaptive visual overlay system for anatomical simulations that modifies wound characteristics responsive to changes in anatomical state, such as limb articulation. U.S. Patent Publication No. 2023/0052960 describes a customizable extended reality patient simulator with rapid case creation tools for healthcare education. U.S. Pat. No. 8,480,403 discloses virtual patient systems using ontological classes with polymesh data mapped to anatomical units for training medical decision-making skills. U.S. Patent Publication No. 2008/0187896 teaches multimodal medical procedure training systems integrating three-dimensional anatomical models with digital video and force-feedback devices.

However, these prior art systems are directed to patient-specific diagnostic reporting, surgical training, wound treatment simulation, or general medical education. None addresses the specific need for visualizing the systemic physiological effects of toxic stress and physical trauma, particularly corporal punishment, in an interactive, educational format accessible to non-medical professionals such as parents, caregivers, and social workers. Furthermore, existing systems lack the combination of medically accurate anatomical modeling, biomarker-driven visualization, spatial audio integration, and cross-platform accessibility necessary for effective toxic stress education.

There exists a critical need for an interactive visualization system that makes the invisible physiological damage of toxic stress visible and comprehensible to diverse audiences, from medical professionals to parents and educators, in order to prevent child maltreatment and improve trauma-informed care practices.

### SUMMARY OF THE INVENTION

The present invention provides a system and method for interactive three-dimensional visualization of physiological stress effects on the human body. The system enables users to observe and understand the complex, multi-system physiological responses triggered by physical trauma and toxic stress events through immersive, medically accurate visualizations accessible via extended reality platforms and conventional display devices.

In accordance with one embodiment, a method for visualizing physiological stress effects comprises: receiving stress event data indicating at least one physical trauma event experienced by a subject; determining, by a processor, a plurality of affected anatomical systems responsive to the stress event data; generating a three-dimensional anatomical model representing at least a portion of the subject's body, the anatomical model comprising volumetric representations of a plurality of organs within the affected anatomical systems; determining biomarker response data indicating physiological changes in the plurality of organs based on the stress event data; modifying visual characteristics of the volumetric representations based on the biomarker response data to depict stress-induced changes; and rendering, on a display device, an interactive three-dimensional visualization comprising the anatomical model with the modified visual characteristics, wherein the visualization is manipulable by user input to view the stress-induced changes from multiple perspectives.

In accordance with another embodiment, a system for visualizing physiological stress effects comprises: a processor; a memory communicatively coupled to the processor and storing executable instructions; a display device communicatively coupled to the processor; and a user input interface. The executable instructions, when executed by the processor, cause the system to: receive stress event data; access anatomical model data representing normal anatomical structures; access biomarker correlation data associating stress events with physiological changes in anatomical structures; generate a three-dimensional anatomical model; determine stress-induced physiological changes based on the stress event data and the biomarker correlation data; modify rendering parameters of the anatomical model to visually depict the stress-induced physiological changes; render an interactive three-dimensional visualization; and update the visualization in real-time responsive to user manipulation via the user input interface.

In accordance with a further embodiment, a non-transitory computer-readable medium contains instructions that, when executed by a processor, cause the processor to perform operations comprising: receiving input data identifying at least one of a demographic characteristic, genetic information, stress event history, and environmental factor associated with a subject; determining, based on the input data, biomarker levels including at least one of cortisol level, blood pressure, blood sugar level, and inflammatory protein level; identifying a plurality of organ systems affected by toxic stress based on the biomarker levels; generating three-dimensional anatomical representations of organs within the identified organ systems; applying visual effects to the three-dimensional anatomical representations to depict stress-related damage; and rendering an interactive visualization comprising the three-dimensional anatomical representations with the applied visual effects on an extended reality device or conventional display.

The system and method may be implemented as a web-based platform compatible with mobile phones, tablets, desktop computers, and extended reality devices including virtual reality headsets and augmented reality glasses. The platform processes medical imaging data, including DICOM format data or other medical imaging formats, using volumetric rendering engines for segmentation and visualization. Suitable volumetric rendering software may include by way of non-limiting example, Slicer3D and Kitware volume viewers, in addition to other medical imaging processing applications. The platform creates optimized three-dimensional anatomical models using modeling software, which may include by way of non-limiting example Autodesk 3DS Max and Blender, though other three-dimensional content creation tools may likewise be used. The platform renders interactive visualizations in real-time using web-based graphics libraries providing abstraction over browser graphics application programming interfaces. Suitable graphics libraries may include, by way of non-limiting example, Three.js, WebGL, WebGPU, or other frameworks. Spatial audio integration provides immersive auditory feedback synchronized with visual representations of physiological processes. Suitable spatial audio technologies may include, by way of non-limiting example, Dolby Atmos, Resonance Audio SDK, or other binaural audio rendering frameworks.

The invention provides particular utility in pediatric toxic stress education, enabling visualization of how corporal punishment affects developing organ systems. Applications include anticipatory guidance in pediatric clinical settings, child protective services training, foster parent education, trauma-informed pedagogy for educators, and curriculum integration in schools of social work, education, and medicine.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1 is a system architecture diagram illustrating the overall system for interactive three-dimensional visualization of physiological stress effects according to certain aspects of an embodiment of the invention.

FIG. 2 is a flowchart illustrating a method for generating an interactive visualization of physiological stress effects according to further aspects of an embodiment of the invention.

FIG. 3 is a schematic representation of the biomarker correlation engine according to further aspects of an embodiment.

FIG. 4 is an exemplary illustration of affected organ systems visualization according to further aspects of an embodiment.

FIG. 5 is an exemplary illustration of a graphical user interface showing an interactive three-dimensional anatomical view according to certain aspects of an embodiment.

FIG. 6 is a schematic diagram illustrating the detailed architecture of audio processing module 600 of FIG. 1 according to certain aspects of an embodiment.

FIG. 7 is a diagram illustrating the detailed architecture of rendering engine module 400 of FIG. 1 according to further aspects of an embodiment.

### DETAILED DESCRIPTION OF THE PREFERRED EMBODIMENTS

The invention may be understood by referring to the following description and accompanying drawings. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

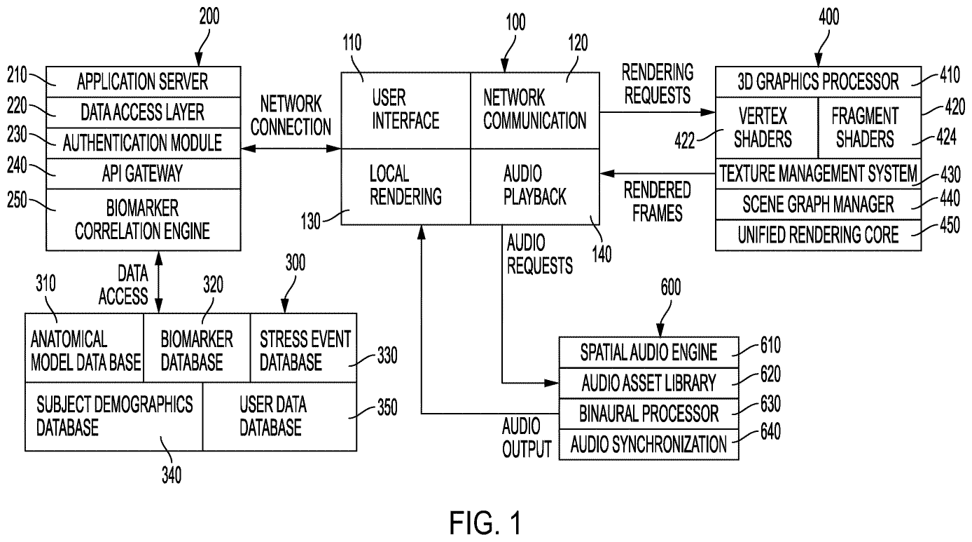

Referring to FIG. 1, the system architecture comprises five interconnected modules: a client application module 100, a web server module 200, a database module 300, a rendering engine module 400, and an audio processing module 600.

The client application module 100 executes on user devices including mobile phones, tablet computers, desktop computers, virtual reality headsets, or augmented reality glasses. The module 100 includes a user interface component 110 for receiving inputs and displaying outputs, a network communication component 120 for data exchange with web server module 200, a local rendering component 130 for graphics processing, and an audio playback component 140 for spatial audio output.

The web server module 200 preferably comprises, by way of non-limiting example, an application server 210 for processing requests, a data access layer 220 for database interaction, an authentication module 230 for access management, an API gateway 240 providing application programming interfaces, such as by way of non-limiting example RESTful interfaces and other web service protocols, and a biomarker correlation engine 250 for determining biomarker levels in response to a subject's particular stress event data.

The database module 300 preferably comprises an anatomical model database 310 storing three-dimensional anatomical structures, a biomarker database 320 storing correlations between stress events and physiological responses, a stress event database 330 storing trauma event definitions, a subject demographics database 340 storing population-specific response data, and a user data database 350 storing user accounts and preferences.

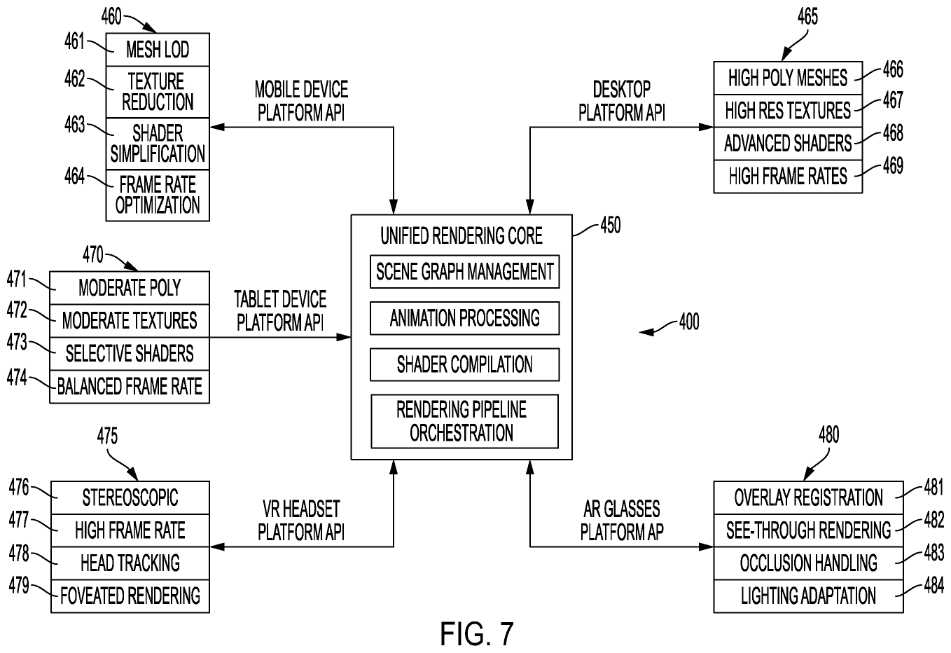

The rendering engine module 400 preferably comprises a 3D graphics processor 410 that, in some exemplary embodiments may implement graphics rendering pipelines for real-time three-dimensional visualization, such as WebGL and WebGPU pipelines, a shader program library 420 containing vertex shaders 422 and fragment shaders 424 for visual effects, a texture management system 430 for texture handling, a scene graph manager 440 for spatial organization of rendered objects, and a unified rendering core 450 providing platform-specific rendering optimization (described in detail below with reference to FIG. 7).

The audio processing module 600 preferably comprises a spatial audio engine 610 for three-dimensional audio positioning, an audio asset library 620 storing physiological sound effects, a binaural processor 630 for immersive audio, and an audio synchronization component 640 for aligning audio with visual animations.

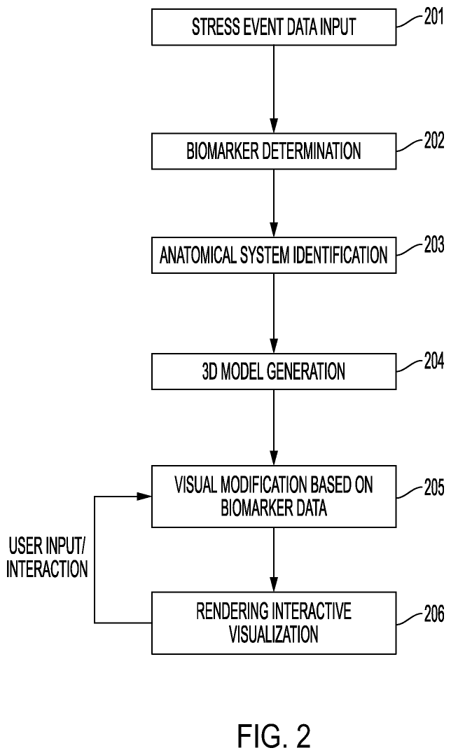

Referring to FIG. 2, a method for generating interactive visualization of physiological stress effects comprises the following steps implemented within the system architecture shown in FIG. 1.

At step 201, stress event data input is received. The system presents an input interface allowing specification of one or more stress events. For corporal punishment, options may specify, for example, hitting method, body location struck, force level, frequency, and subject age. The stress event data is stored in stress event database 330 (FIG. 1).

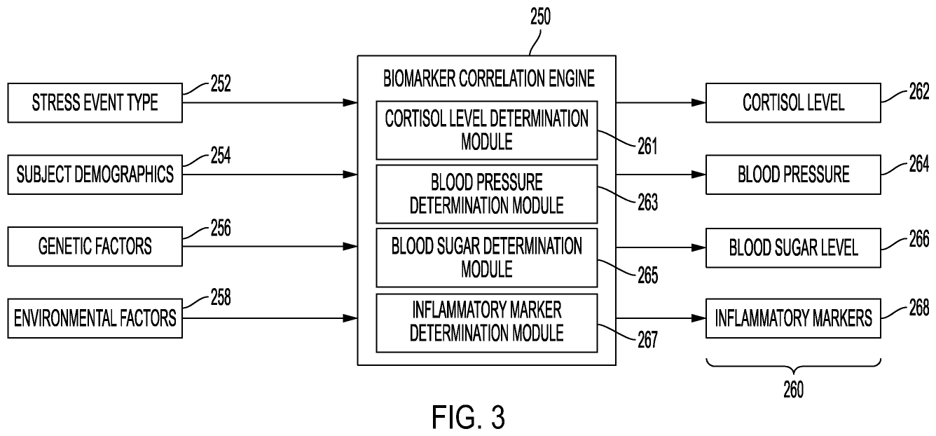

At step 202, biomarker determination is performed using biomarker correlation engine 250 (described below with reference to FIG. 3). The engine 250 retrieves stress event data 252 and associated subject demographics 254, genetic factors 256, and environmental factors 258 from respective databases (FIG. 1), applying correlation algorithms to determine biomarker response levels 260 including cortisol 262, blood pressure 264, blood sugar 266, and inflammatory markers 268.

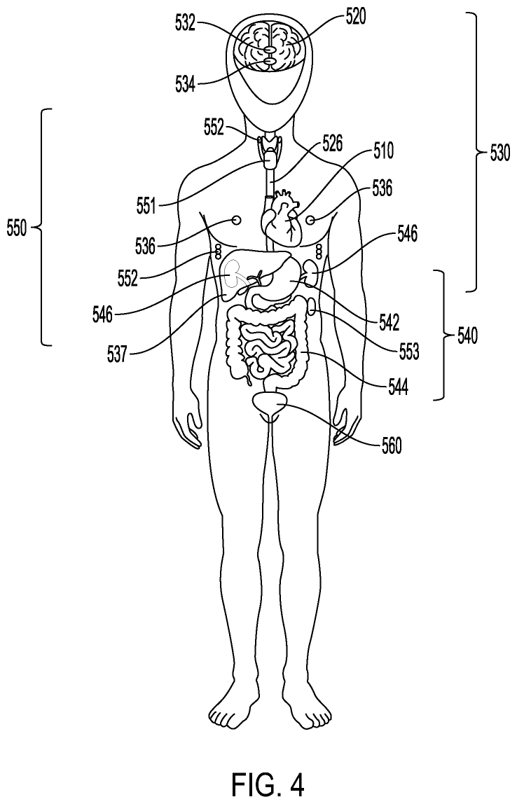

At step 203, anatomical system identification is performed. Based on determined biomarker levels 260, the system identifies organ systems exhibiting significant physiological changes using threshold-based selection. For example, elevated cortisol may trigger endocrine system 530 selection (described below with reference to FIG. 4), elevated inflammatory markers may trigger immune system 550 selection, and elevated blood pressure may trigger cardiovascular system 510 selection.

At step 204, three-dimensional model generation retrieves base anatomical models from anatomical model database 310 (FIG. 1) representing normal, healthy anatomical structures corresponding to organ systems, loading them into rendering engine module 400.

At step 205, visual modification applies modifications to base anatomical models based on biomarker response data from step 202, creating visual indicators. Color modification algorithms may alter texture maps managed by texture management system 430 (FIG. 1). For example, inflamed tissues may render with increased red channel values, ischemic tissues with decreased saturation, and hypertrophied tissues with modified specular properties. Geometric modification algorithms may deform polygon meshes. For example, cardiac hypertrophy may be represented by increased ventricular wall thickness in cardiovascular system 510, vascular damage by surface irregularities, and atrophied structures by reduced scale in nervous system 520. Further, animated effects may depict dynamic processes. For example, rapid heartbeat may be depicted via accelerated contraction animations, elevated blood flow via increased particle speeds, and neural pathway disruption via intermittent connections in nervous system 520.

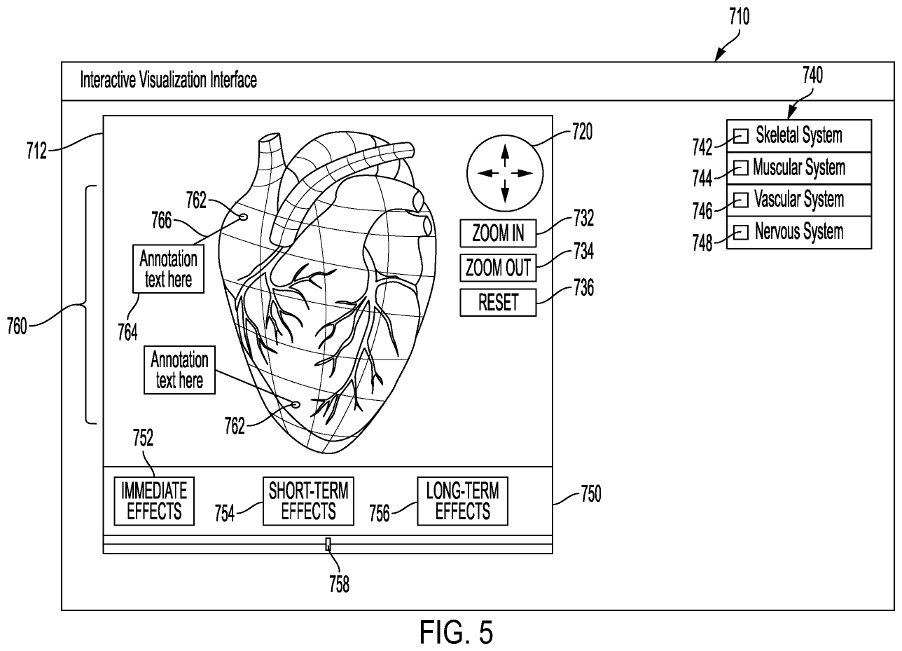

Temporal state selected via temporal progression controls 750 (described below with reference to FIG. 5) may determine modification magnitude, with immediate effects producing subtle changes, short-term effects producing moderate changes, and long-term effects producing pronounced changes. Anatomical layer toggles 740 (FIG. 5) control transparency and layering. Visual effects are parameterized based on biomarker magnitude.

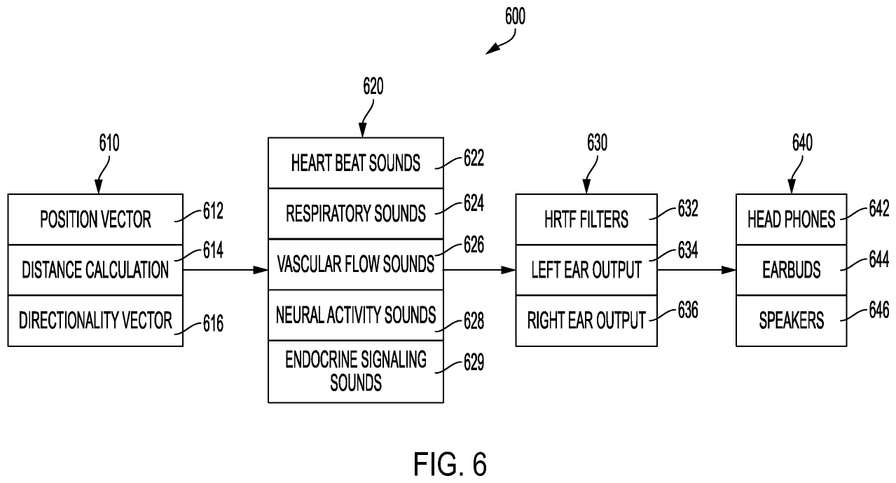

At step 206, rendering is performed via rendering engine module 400 (FIG. 1), processing modified anatomical models through the graphics pipeline to generate displayable images in viewport 712. Rendering utilizes the appropriate platform-specific rendering module (FIG. 7) based on device type, executing vertex transformation using vertex shaders 422, lighting calculations, texture sampling from texture management system 430, fragment shading using fragment shaders 424, and post-processing effects. Frames transmit to the display device of client application module 100 (FIG. 1). Simultaneously, audio processing module 600 (described below with reference to FIG. 6) generates corresponding audio synchronized via audio synchronization component 640 (FIG. 1).

The rendering loop preferably executes continuously, responding to user inputs via rotation controls 720, zoom controls 732, 734, and 736, anatomical layer toggles 740, and temporal progression controls 750. User interactions are processed by user interface component 110 (FIG. 1), and transmitted to rendering engine module 400, which recalculates scene parameters and re-renders updated frames.

Referring to FIG. 3, the biomarker correlation engine 250 processes multiple inputs to determine physiological stress responses. The engine receives stress event type data 252 identifying the nature, severity, frequency, and duration of physical trauma or toxic stress exposure. Subject demographics data 254 provides age, gender, developmental stage, and background information that influence stress responses. Genetic factors data 256 indicates genetic predispositions affecting stress sensitivity. Environmental factors data 258 captures social support systems, prior trauma exposure, and protective factors.

The biomarker correlation engine 250 applies correlation algorithms comprising rule-based systems, statistical models, or machine learning models trained on medical research data to determine biomarker levels 260. Cortisol level determination module 261 calculates cortisol level values 262 including HPA axis activation and cortisol elevations. Blood pressure determination module 263 calculates blood pressure values 264 including sympathetic nervous system activation and cardiovascular responses. Blood sugar determination module 265 calculates blood sugar levels 266 indicating metabolic disruptions. Inflammatory marker determination module 267 calculates inflammatory markers 268 including elevations in C-reactive protein, interleukin-6, tumor necrosis factor-alpha, and other inflammatory mediators.

The determined biomarker levels may include temporal progression data indicating immediate responses (minutes to hours), short-term responses (days to weeks), and long-term responses (months to years), enabling visualization of both acute stress responses and chronic allostatic load effects via the temporal progression controls 750 shown in FIG. 5.

Referring to FIG. 4, the system generates visualizations across multiple organ systems, each represented by three-dimensional anatomical models with visual indicators depicting stress-related changes.

The cardiovascular system 510 comprises models of the heart, blood vessels, and microvascular networks. Visual indicators may depict, for example, increased heart rate (via animation speed), elevated blood pressure (via vessel wall thickness and color intensity), endothelial dysfunction (via surface texture modifications), and atherosclerotic changes (via geometric protrusions and color variations).

The nervous system 520 comprises models of the brain, spinal cord, peripheral nerves, and vagus nerve 526. Visual indicators may depict, for example, amygdala activation (via color intensity and pulsing), hippocampal volume reduction (via geometric scaling), prefrontal cortex connectivity disruption (via broken neural pathway visualizations), autonomic dysregulation (via animated signals along vagus nerve 526), and neurotransmitter imbalances (via particle effects at synapses).

The endocrine system 530 comprises models of hypothalamus 532, pituitary 534, adrenal glands 536, thyroid, and pancreas 537. Visual indicators may depict, for example, HPA axis hyperactivation (via animated signaling cascade from hypothalamus 532 through pituitary 534 to adrenal cortex 536), glucocorticoid receptor changes, thyroid dysfunction, and early puberty triggering.

The digestive system 540 comprises models of stomach 542, intestines 544, liver, and kidneys 546. Visual indicators may depict, for example, gastric acid dysregulation, intestinal permeability increases, kidney stress responses, and enzyme imbalances.

The immune system 550 comprises models of thymus 551, spleen 553, lymph nodes 552, and bone marrow. Visual indicators may depict, for example, chronic inflammation (via elevated color temperature and cytokine particle effects), immune cell dysfunction (via altered animations in lymphoid organs), and infection susceptibility (via pathogen invasion visualizations).

The reproductive system 560 comprises age-appropriate reproductive organ models. Visual indicators may depict, for example, hormonal disruption, early puberty onset, and reproductive dysfunction.

Referring to FIG. 5, the interactive three-dimensional anatomical view 710 provides the primary visualization interface. Central viewport 712 renders the three-dimensional anatomical models (FIG. 4) with stress-related modifications, supporting real-time rotation, panning, and zooming.

Rotation controls 720 may enable rotation about three axes via on-screen widgets, gesture inputs, or head-tracking for VR/AR devices. The system maintains smooth frame rates (60+ fps for desktop, 90+ fps for VR) to prevent user discomfort.

Zoom controls adjust viewing distance and field of view via zoom in button 732, zoom out button 734, and reset view button 736.

Anatomical layer toggles 740 may enable selective display of structures via skeletal system toggle 742, muscular system toggle 744, vascular system toggle 746, nervous system toggle 748, and additional toggles for other organ systems, allowing progressive revelation of internal structures.

Temporal progression controls 750 enable visualization of stress effects across different time scales. For example, immediate effects button 752 configures the visualization to depict physiological changes occurring within minutes to hours after a stress event. Short-term effects button 754 configures the visualization to depict changes occurring over days to weeks. Long-term effects button 756 configures the visualization to depict chronic changes occurring over months to years. Animated timeline slider 758 may allow continuous navigation through the temporal progression, with the anatomical models updating in real-time to show time-dependent changes.

The temporal progression feature visualizes physiological changes by varying the intensity of visual indicators in the anatomical models based on the selected temporal state. When immediate effects button 752 is activated, the anatomical models render with modest visual indicator intensity representing acute stress responses occurring within minutes to hours: for example, cardiovascular system 510 displays rapid heart rate increase, nervous system 520 shows amygdala activation, and endocrine system 530 illustrates initial cortisol release from adrenal glands 536. When short-term effects button 754 is activated, visual indicators increase in intensity to represent changes occurring over days to weeks: for example, cardiovascular system 510 displays sustained elevated blood pressure with vessel wall thickening, immune system 550 shows inflammatory marker elevation, digestive system 540 displays gastrointestinal disruption in stomach lining 542 and intestinal wall 544. When long-term effects button 756 is activated, visual indicators display maximum intensity representing chronic changes over months to years: for example, nervous system 520 shows structural brain changes including hippocampal volume reduction and disrupted neural connectivity, cardiovascular system 510 displays atherosclerotic changes, endocrine system 530 shows HPA axis dysregulation with chronically enlarged adrenal glands 536. Animated timeline slider 758 allows continuous navigation between these temporal states, with rendering engine module 400 (FIG. 1) progressively updating visual characteristics in real-time based on biomarker temporal progression data from biomarker correlation engine 250. The temporal progression data is determined based on medical research correlating stress exposure duration with biomarker evolution: cortisol 262 peaks within hours and normalizes within days for acute stress, inflammatory markers 268 remain elevated for weeks or months, structural organ changes manifest over months or years.

Annotation overlays 760 provide educational information via annotation markers 762 positioned at anatomical locations, connected to information callouts 764 via leader lines 766, displaying text describing physiological processes.

Referring to FIG. 6 (providing a detailed view of the audio processing module 600 of FIG. 1), the audio processing module 600 provides immersive auditory feedback synchronized with visual animations. Audio processing module 600 preferably provides both narration and physiological sound effects synchronized with visual animations, creating a multimodal sensory experience reinforcing understanding of internal physiological processes.

The spatial audio engine 610 determines three-dimensional positions of audio sources relative to the listener's viewpoint, calculating position vector 612, distance 614, and directionality vector 616 for each active source. These parameters update in real-time as users manipulate the view via rotation controls 720 and zoom controls 732, 734, and 736.

Audio asset library 620 may comprise physiological sounds including heartbeat sounds 622, respiratory sounds 624, vascular flow sounds 626, neural activity sounds 628, and endocrine signaling sounds 629.

The binaural processor 630 may apply head-related transfer functions via HRTF filters 632 to generate three-dimensional audio for headphone playback, processing sources to create left ear 634 and right ear 636 signals that simulate sounds emanating from specific spatial locations. For speaker-based playback, multi-channel surround sound processing including, for example, Dolby Atmos rendering may be employed. The spatial audio engine 610 (FIG. 1) implements spatial audio processing libraries providing cross-platform three-dimensional audio positioning and binaural rendering. Suitable spatial audio libraries include, by way of non-limiting example, Resonance Audio SDK or other binaural audio rendering frameworks supporting real-time head-related transfer function processing.

Audio synchronization component 640 outputs to audio devices comprising headphones 642, earbuds 644, or speaker systems 646. The audio synchronization component 640 (FIG. 1) ensures temporal alignment between audio events and visual animations, such that heartbeat sounds coincide with heart contractions in cardiovascular system 510 (FIG. 4).

Referring to FIG. 7, the rendering engine module 400 (introduced in FIG. 1) enables consistent visualization across diverse hardware while maintaining visual fidelity of the above-described anatomical models and interactive features. Unified rendering core 450 implements platform-agnostic rendering logic including scene graph management, animation processing, shader compilation, and rendering pipeline orchestration.

Mobile device rendering module 460 optimizes for smartphones via mesh level-of-detail reduction 461, texture resolution adaptation 462, shader complexity reduction 463, and frame rate optimization 464. For mobile web browsers, the module implements browser-based graphics APIs which may include, by way of non-limiting example, WebGL, WebGPU, or successor mobile browser graphics standards. For native mobile applications, the module may implement native graphics APIs including, by way of non-limiting example, Metal, Vulkan, or other platform-specific graphics frameworks.

Desktop browser rendering module 465 leverages higher processing capabilities for higher polygon count meshes 466, higher resolution textures 467, advanced shader effects 468 (e.g., subsurface scattering, ambient occlusion), and higher frame rates 469 (60+ fps). The module implements browser graphics APIs with automatic selection based on browser capabilities. Suitable graphics APIs include, by way of non-limiting example, WebGL, WebGPU, or future browser-native graphics standards providing GPU-accelerated rendering.

Tablet device rendering module 470 provides intermediate optimization with moderate polygon counts 471, moderate texture resolutions 472, selective advanced shaders 473, and balanced frame rates 474.

VR headset rendering module 475 addresses virtual reality requirements via stereoscopic rendering 476 (separate left/right eye images), high frame rate maintenance 477 (minimum 90 fps), low-latency head tracking integration 478, and foveated rendering 479 (higher quality at gaze center, reduced quality in peripheral vision).

AR glasses rendering module 480 addresses augmented reality requirements via real-world overlay registration 481, optical see-through or video see-through rendering 482, occlusion handling 483, and environmental lighting adaptation 484.

In an exemplary configuration, the unified rendering core 450 utilizes a web-based three-dimensional graphics rendering library providing abstraction over underlying browser graphics application programming interfaces. In certain exemplary configurations, the graphics rendering library may comprise JavaScript-based libraries such as by way of non-limiting example Three.js, Babylon.js, or other frameworks compatible with browser-based graphics APIs. The underlying graphics APIs may include WebGL, WebGPU, or successor browser-native graphics standards as they become available. The rendering pipeline supports physically-based rendering (PBR) materials with base color, metallic/roughness, normal maps, and emissive channels. The animation system interpolates anatomical deformations, color transitions, and other visual changes over time to depict physiological processes across the temporal progression states controlled via temporal progression controls 750.

Three-dimensional anatomical models stored in anatomical model database 310 (FIG. 1) and rendered as organ systems (FIG. 4) are preferably created from medical imaging data. Medical imaging files, including but not limited to DICOM files containing CT or MRI scan data, may be processed using volumetric processing software for segmentation and isosurface extraction. Suitable volumetric processing libraries include, by way of non-limiting example, Slicer3D, Kitware VTK, or other medical imaging processing frameworks supporting isosurface generation and mesh extraction. Segmentation algorithms delineate anatomical structure boundaries. Marching cubes algorithms generate polygon meshes comprising vertices, edges, and faces. Mesh optimization via decimation, smoothing, and topology correction creates different polygon count levels for platform-specific rendering: higher polygon meshes 466 for desktop, moderate polygon counts 471 for tablet, reduced meshes via mesh LOD reduction 461 for mobile. Texture mapping applies UV coordinates associating vertices with texture images (color maps, normal maps, specular maps, opacity maps) managed by texture management system 430 (FIG. 1) and adapted via texture resolution adaptation 462 for mobile or higher resolution textures 467 for desktop (FIG. 7). Three-dimensional polygon mesh modeling software may serve as primary modeling tools for medical artists creating anatomically accurate organ models. Suitable modeling software includes, by way of non-limiting example, Autodesk 3DS Max, Blender, or other digital content creation applications supporting polygon mesh modeling, texture mapping, and anatomical sculpting.

The system implements web-based technologies for cross-platform deployment. Client-side application within client application module 100 (FIG. 1) utilizes web technologies including markup languages (e.g., HTML5), styling languages (e.g., CSS3), and client-side scripting languages (e.g., JavaScript). Three-dimensional graphics are provided via a graphics rendering library (described above with reference to unified rendering core 450) interfacing with browser graphics APIs. User interface components are managed using declarative UI frameworks, which may include by way of non-limiting example React, Vue.js, or other component-based UI libraries. Server-side implementation utilizes a server-side runtime environment for application server 210 (FIG. 1) providing API endpoints through API gateway 240. Suitable server platforms include, by way of non-limiting example, Node.js, Python-based frameworks, Java-based frameworks, or other server-side application platforms supporting web service development. Database storage for database module 300 implements database management systems suitable for structured data, medical imaging metadata, and user information. Suitable relational databases include, by way of non-limiting example, PostgreSQL, MySQL, or other SQL-compliant database systems. For flexible storage, suitable NoSQL databases may include, by way of non-limiting example, MongoDB or other document-oriented or key-value database systems. Spatial audio processing utilizes professional audio production software for audio asset creation and mixing. Suitable audio production tools may include, by way of non-limiting example, Pro Tools or other digital audio workstations. For spatial audio encoding, suitable technologies may include, by way of non-limiting example, Dolby Atmos or other immersive audio formats. For real-time binaural rendering, suitable spatial audio libraries may include, by way of non-limiting example, Resonance Audio SDK or other binaural audio rendering frameworks integrated into audio playback component 140 (FIG. 1) via binaural processor 630.

User input detection captures events from input devices connected to user interface component 110 (FIG. 1). Touch events, mouse inputs, VR/AR controller inputs, and voice commands are classified into interaction categories: rotation interactions affecting rotation controls 720 adjust camera viewpoint around anatomical models in viewport 712; zoom interactions via zoom controls 732, 734 adjust camera distance; reset view button 736 returns to default viewpoint; selection interactions identify anatomical structures for annotation display via annotation markers 762 and information callouts 764; toggle interactions via anatomical layer toggles 740 show/hide organ systems; temporal navigation via temporal progression controls 750 adjusts between temporal states. Model transformation calculation by scene graph manager 440 (FIG. 1) computes updated scene parameters. Re-rendering by rendering engine module 400 using the appropriate platform module generates updated frames at frame rates determined by platform: frame rate optimization 464 for mobile devices, higher frame rates 469 for desktop browsers, balanced frame rates 474 for tablets, high frame rate maintenance 477 for VR headsets.

In an exemplary, hypothetical pediatric corporal punishment education scenario, a healthcare provider accesses the platform on a tablet device utilizing tablet device rendering module 470 (FIG. 7) with moderate polygon counts 471 and moderate texture resolutions 472. The provider inputs stress event data at step 201 of the method described above specifying a 4-year-old child and “corporal punishment—spanking” as stress event type 252 (FIG. 3). Biomarker correlation engine 250 processes this with subject demographics 254 and determines biomarker levels 260 at step 202. The system loads a pediatric-scaled anatomical model in central viewport 712.

Using anatomical layer toggles 740, the provider selects nervous system toggle 748, highlighting nervous system 520 from FIG. 4 in viewport 712. Activating immediate effects button 752 displays physiological changes occurring within minutes to hours, showing amygdala activation via visual indicators, stress hormone cascade in endocrine system 530, and autonomic dysregulation along vagus nerve 526.

Audio processing module 600 (FIG. 6) may include heartbeat sounds 622 spatially positioned at cardiovascular system 510, processed through binaural processor 630 via HRTF filters 632, output as left ear 634 and right ear 636 signals to headphones 642, earbuds 644 or speaker systems 646. Narration from audio asset library 620 explains the physiological cascade.

The provider activates long-term effects button 756 via animated timeline slider 758. Visual modification step 205 updates models to show hippocampal volume reduction, chronically enlarged adrenal glands 536 in endocrine system 530 with intensified visual indicators, and immune system 550 suppression with modified visual indicators. Annotation overlays 760 provide scientific context via annotation markers 762, leader lines 766, and information callouts 764 in viewport 712.

The present invention provides specific technological solutions to technical problems in medical visualization and education. The invention particularly implements concrete technical transformations: (1) processing stress event data and biomarker correlation data through specialized algorithms (biomarker correlation engine 250) to generate specific physiological response predictions; (2) transforming these predictions into visual modification parameters affecting polygon mesh geometry, texture properties, animation parameters, and transparency values; (3) rendering these modifications through platform-specific graphics pipelines using shader programs 422, 424 and texture management system 430; and (4) generating synchronized spatial audio through binaural processor 630 using HRTF filters 632.

The invention improves computer visualization technology itself by providing novel combinations of biomarker-driven real-time visual modification of anatomical models, temporal progression visualization enabling dynamic interpolation between physiological states via temporal progression controls 750, multi-system integrated visualization across multiple organ systems simultaneously, spatial audio synchronized with visual physiological processes, and cross-platform adaptive rendering optimizing visualization for diverse hardware capabilities.

These technical features offer improvements over previously known anatomical visualization systems. Unlike patient-specific diagnostic visualization (U.S. Pat. No. 12,308,099) requiring actual patient data, the invention uses population-level biomarker correlations enabling anticipatory educational guidance. Moreover, unlike anatomical training simulators (U.S. Pat. Nos. 11,557,216, 8,480,403, Publication No. 2008/0187896) focused on procedural skills for medical professionals, the invention provides toxic stress-specific educational visualizations accessible to non-medical audiences via web-based cross-platform deployment. The unique combination of biomarker-driven visualization, temporal progression, multi-system integration, spatial audio, and cross-platform accessibility addresses unmet needs in toxic stress education.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. Thus, it should be understood, therefore, that the invention may be practiced otherwise than as specifically set forth herein.

What is claimed is:

1. A computer-implemented method for visualizing physiological stress effects, comprising:
receiving, by a processor, stress event data indicating at least one physical trauma event;
determining, by the processor, a plurality of affected anatomical systems responsive to the stress event data, the affected anatomical systems selected from a group consisting of a cardiovascular system, a nervous system, an endocrine system, a digestive system, an immune system, and a reproductive system;
generating, by the processor, a three-dimensional anatomical model representing at least a portion of a subject's body, the anatomical model comprising volumetric representations of a plurality of organs within the affected anatomical systems;
determining, by the processor, biomarker response data indicating physiological changes in the plurality of organs based on the stress event data, the biomarker response data including at least one biomarker selected from cortisol level, blood pressure, blood sugar level, and inflammatory protein level;
modifying, by the processor, visual characteristics of the volumetric representations based on the biomarker response data to depict stress-induced changes in the plurality of organs; and
rendering, on a display device, an interactive three-dimensional visualization comprising the anatomical model with the modified visual characteristics, wherein the visualization is manipulable by user input to view the stress-induced changes from multiple perspectives.

2. The method of claim 1, wherein the stress event data comprises data indicating corporal punishment of a pediatric subject, and wherein the anatomical model is scaled to represent pediatric anatomical proportions.

3. The method of claim 1, wherein determining biomarker response data further comprises:
accessing a biomarker correlation database storing associations between stress event types and physiological biomarker changes; and
applying correlation algorithms to the stress event data using the associations to calculate expected biomarker levels.

4. The method of claim 1, wherein modifying visual characteristics comprises at least one modification selected from the group consisting of: altering color values of texture maps applied to the volumetric representations, deforming polygon mesh geometry of the volumetric representations, adjusting transparency values of the volumetric representations, and generating animated visual effects depicting dynamic physiological processes.

5. The method of claim 1, further comprising:
receiving temporal selection input indicating a selected time period from a group consisting of immediate effects, short-term effects, and long-term effects; and
modifying the visual characteristics based on the temporal selection input to depict physiological changes corresponding to the selected time period.

6. The method of claim 1, wherein rendering the interactive three-dimensional visualization comprises rendering for an extended reality device selected from the group consisting of a virtual reality headset and augmented reality glasses.

7. The method of claim 1, further comprising:
generating spatial audio data representing physiological sounds associated with the affected anatomical systems;
processing the spatial audio data through binaural audio rendering to position audio sources in three-dimensional space corresponding to locations of the plurality of organs in the anatomical model; and
outputting the spatial audio synchronously with the interactive three-dimensional visualization.

8. A system for interactive visualization of toxic stress effects on anatomical structures, comprising:
a processor;
a memory communicatively coupled to the processor and storing executable instructions and anatomical model data;
a display device communicatively coupled to the processor;
a user input interface communicatively coupled to the processor; and
an audio output device communicatively coupled to the processor;
wherein the executable instructions, when executed by the processor, cause the system to:
receive input data identifying at least one characteristic of a subject selected from demographic information, genetic information, stress event history, and environmental factors;
access the anatomical model data comprising three-dimensional polygon mesh representations of human organs created using three-dimensional modeling software;
access biomarker correlation data associating stress events with physiological changes in anatomical structures;
process medical imaging data in DICOM format using volumetric rendering software to generate anatomical structure representations;
generate a three-dimensional interactive anatomical visualization by:
determining stress-induced physiological changes in a plurality of organ systems based on the input data and the biomarker correlation data,
applying rendering transformations to the polygon mesh representations using a web-based graphics rendering library to visually depict the stress-induced physiological changes with modified color, geometry, transparency, or animation parameters, and
compositing the transformed polygon mesh representations into a unified three-dimensional scene;
generate spatial audio using a spatial audio processing library, the spatial audio comprising physiological sounds positioned in three-dimensional space corresponding to locations of organs in the three-dimensional scene;
render the three-dimensional interactive anatomical visualization on the display device using a rendering pipeline comprising vertex processing, fragment shading, and post-processing effects;
output the spatial audio through the audio output device synchronously with the rendered visualization; and
update the visualization in real-time responsive to manipulation inputs received via the user input interface.

9. The system of claim 8, wherein the three-dimensional modeling software comprises a polygon mesh modeling application supporting import of medical imaging segmentation data and providing sculpting tools for anatomical detail refinement, wherein the modeling software exports anatomical models in web-compatible three-dimensional formats.

10. The system of claim 8, wherein the web-based graphics rendering library executes within a web browser environment without requiring plugin installation and implements a scene graph architecture for hierarchical organization of anatomical model components.

11. The system of claim 8, wherein the volumetric rendering software processes medical imaging data, performs automated segmentation to identify anatomical structure boundaries, and generates polygon meshes from volumetric imaging data to create anatomical models suitable for real-time web-based rendering.

12. The system of claim 8, wherein the spatial audio processing library applies head-related transfer functions to audio source signals based on three-dimensional position coordinates of anatomical organs relative to a listener position.

13. The system of claim 8, wherein the executable instructions further cause the system to:
provide a graphical user interface comprising selectable anatomical system icons each representing one of the plurality of organ systems;
receive selection input indicating a selected anatomical system icon; and
configure the three-dimensional interactive anatomical visualization to emphasize volumetric representations associated with the selected anatomical system while rendering other anatomical structures with reduced opacity.

14. The system of claim 8, wherein the display device comprises a device selected from the group consisting of a mobile phone screen, a tablet computer screen, a desktop computer monitor, a virtual reality headset display, and augmented reality glasses.

15. A non-transitory computer-readable medium storing instructions that, when executed by a processor, cause the processor to perform operations comprising:
receiving stress event input data identifying corporal punishment event parameters including body location struck, force level, and frequency;
receiving subject age data indicating a pediatric subject age;
accessing an anatomical structure database comprising three-dimensional polygon mesh models of pediatric organs including a brain model, a heart model, adrenal gland models, and a vagus nerve model, wherein each polygon mesh model comprises vertices, edges, faces, and associated UV texture coordinates;
calculating cortisol level elevation based on the corporal punishment event parameters and the subject age using a biomarker calculation algorithm;
calculating cardiovascular response parameters including heart rate increase and blood pressure elevation based on the corporal punishment event parameters and the subject age;
identifying affected organ systems comprising at least a nervous system and an endocrine system based on the calculated cortisol level elevation and cardiovascular response parameters exceeding predefined threshold values;
loading polygon mesh models for organs within the identified affected organ systems from the anatomical structure database;
generating visual modification parameters comprising:
color shift parameters for texture maps of the adrenal gland models representing hyperactivation state,
geometry scaling parameters for the heart model representing increased cardiac workload,
animation speed parameters for heartbeat animation representing elevated heart rate, and
transparency parameters for neural pathway representations in the brain model representing connectivity disruption;
applying the visual modification parameters to the loaded polygon mesh models using shader programs executed on a graphics processing unit;
rendering a three-dimensional scene comprising the modified polygon mesh models using a hardware-accelerated browser graphics rendering pipeline executing in a web browser environment;
generating spatial audio comprising:
a heartbeat sound effect positioned at three-dimensional coordinates of the heart model and played at a tempo corresponding to the calculated heart rate increase, and
a narration audio track explaining physiological stress responses;
processing the spatial audio through binaural rendering using head-related transfer functions to generate left channel audio and right channel audio;
displaying the rendered three-dimensional scene on a display;
outputting the left channel audio and right channel audio through stereo headphones;
detecting user input via a touch interface, mouse interface, or controller interface;
updating camera position or camera orientation in the three-dimensional scene responsive to the detected user input; and
re-rendering the three-dimensional scene with the updated camera position or camera orientation to provide real-time interactive manipulation.

16. The non-transitory computer-readable medium of claim 15, wherein the operations further comprise:
receiving temporal mode selection input indicating selection from a group consisting of immediate effects mode, short-term effects mode, and long-term effects mode;
accessing temporal progression data associating time periods with physiological change magnitudes; and
adjusting the visual modification parameters based on the temporal mode selection and the temporal progression data, wherein long-term effects mode produces greater magnitude modifications than immediate effects mode.

17. The non-transitory computer-readable medium of claim 15, wherein generating spatial audio further comprises:
loading physiological sound assets from an audio library, the physiological sound assets comprising pre-recorded or synthesized sounds representing heart sounds, blood flow sounds, and neural activity sounds;
determining three-dimensional position coordinates for each physiological sound asset based on anatomical positions of corresponding organs in the three-dimensional scene; and
calculating distance attenuation and directional filtering for each physiological sound asset based on a listener position corresponding to a camera position in the three-dimensional scene.

18. The non-transitory computer-readable medium of claim 15, wherein the biomarker calculation algorithm comprises a rule-based algorithm, a statistical regression model, or a machine learning model trained on medical research data correlating corporal punishment parameters with measured biomarker responses in pediatric populations.

19. The non-transitory computer-readable medium of claim 15, wherein the operations further comprise:
determining a subject demographic category based on the subject age data and additional demographic data; and
retrieving population-specific biomarker correlation data corresponding to the subject demographic category;
wherein calculating cortisol level elevation and calculating cardiovascular response parameters utilize the population-specific biomarker correlation data to provide demographic-adjusted physiological response predictions.

20. The non-transitory computer-readable medium of claim 15, wherein rendering the three-dimensional scene comprises:
performing platform detection to identify a device type selected from a mobile device, a desktop device, and a virtual reality device;
selecting a level-of-detail configuration for the polygon mesh models based on the identified device type, wherein mobile device selection results in reduced polygon counts and desktop device selection results in higher polygon counts; and
rendering the polygon mesh models using the selected level-of-detail configuration to optimize performance for the identified device type.
